(19) United States
(12) Patent Application Publication
(10) Pub. No.: US 2007/0066985 A1
(43) Pub. Date: Mar. 22, 2007

(54) Tissue cutting devices having hemostasis capability and related methods

(75) Inventors: Kurt A.E. Geitz, Sudbury, MA (US); David Hoffman, Concord, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: BOSTON SCIENTIFIC SCIMED, INC., Maple Grove, MN (US)

(21) Appl. No.: 11/227,284

(22) Filed: Sep. 16, 2005

(51) Int. Cl. A61B 17/32

(52) U.S. Cl. 606/170

(57) ABSTRACT

Various embodiments of a medical device (e.g., tissue acquisition device) having a separate hemostasis capability and related methods of use are disclosed. The device may include an elongated tubular member having a proximal end and a distal end, a tissue cutting member proximate the distal end of the tubular member, and a hemostatic member proximate the distal end of the tubular member and adjacent the tissue cutting member. At least one of the tissue cutting member and the hemostatic member may be fixedly connected to the tubular member.

### FIELD OF THE INVENTION

The present invention relates generally to medical devices and related methods. More specifically, particular embodiments of the invention relate to tissue acquisition devices (e.g., biopsy forceps) having a separate hemostasis capability for use in, for example, acquiring tissue samples from a body.

### DESCRIPTION OF RELATED ART

Biopsy forceps are widely used to obtain tissue samples. A biopsy forceps generally includes a distal cutting member configured to sever tissue samples and a tissue receptacle for holding one or more of the severed tissue samples. Severing tissue from a tissue site causes that tissue site to bleed. To stop or minimize the bleeding during the procedure, “hot” biopsy forceps may be used to direct cauterizing electric current or thermal energy to the tissue site through the distal cutting member to cauterize the bleeding tissue site. Such “hot” biopsy forceps, however, may damage the tissue samples collected in the biopsy forceps, such that a proper diagnosis of the tissue samples may be difficult.

Thus, there is a need for biopsy forceps having a separate hemostasis capability, which does not damage the tissue sample without compromising the tissue acquisition and/or hemostasis capabilities.

### SUMMARY OF THE INVENTION

Therefore, various exemplary embodiments of the invention may provide biopsy forceps having a separate hemostatic device integrated with the biopsy forceps, which may effectively stop the bleeding at the tissue site without damaging the tissue samples obtained by the biopsy forceps.

To attain the advantages and in accordance with the purpose of the invention, as embodied and broadly described herein, one aspect of the invention may provide a medical device comprising an elongated tubular member having a proximal end and a distal end, a tissue cutting member proximate the distal end of the tubular member, and a hemostatic member proximate the distal end of the tubular member and adjacent the tissue cutting member. In some exemplary aspects, at least one of the tissue cutting member and the hemostatic member may be fixedly connected to the tubular member.

According to another exemplary aspect of the invention, a method of performing a medical procedure may be provided. The method may comprise inserting a medical device into a patient's body, where the medical device may comprise an elongated tubular member having a proximal end and a distal end, a tissue cutting member proximate the distal end of the tubular member, and a hemostatic member proximate the distal end of the tubular member and adjacent the tissue cutting member. At least one of the tissue cutting member and the hemostatic member may be fixedly connected to the tubular member. The method may also include positioning the distal end of the tubular member near a target site, severing tissue from the target site with the tissue cutting member, and stopping bleeding at the target site with the hemostatic member. In an aspect, the method may further comprise removing the medical device out of the patient's body. In another aspect, stopping bleeding may comprise moving the hemostatic member axially relative to the tissue cutting member.

According to another aspect, both of the tissue cutting member and the hemostatic member may be fixedly connected to the tubular member. In various other aspects, one of the tissue cutting member and the hemostatic member may be fixedly connected to the tubular member, and the other of the tissue cutting member and the hemostatic member may be movably arranged within the tubular member. In one aspect, the tubular member may define a lumen extending between the proximal end and the distal end, and the lumen may be configured to accommodate the other of the tissue cutting member and the hemostatic member.

In some aspects of the invention, the tissue cutting member may comprise a tissue acquisition device. The tissue acquisition device may comprise a biopsy forceps. Alternatively or additionally, the tissue cutting member may comprise at least one of a snare, scissors, pincer, needle, knife, needleknife, and basket.

In another aspect, the hemostatic member may comprise one of a laser fiber probe, a heater probe, an injection needle configured to inject a therapeutic agent, an electrocautery device, a clip applicator, a ligation device configured to apply a band or loop, and a gas-induced plasma probe.

According to one aspect of the invention, the elongated member may be sufficiently flexible to traverse tortuous anatomy of a patient.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate an exemplary embodiment of the invention and together with the description, serve to explain the principles of the invention. In the drawings:

FIG. 1 is a schematic view of a biopsy forceps having separate hemostasis capability, according to an exemplary embodiment of the invention;

FIG. 2 is a partial schematic view of the distal portion of the biopsy forceps shown in FIG. 1;

FIGS. 3-5 are schematic cross-sectional views of the biopsy forceps shown in FIG. 2, along III-III plane, according to various exemplary embodiments of the invention;

FIG. 6 is a partial schematic view of a hemostatic device, according to another exemplary embodiment of the invention;

FIG. 7 is a partial schematic view of a hemostatic device, according to still another exemplary embodiment of the invention;

FIG. 8 is a partial schematic view of a hemostatic device, according to still yet another exemplary embodiment of the invention; and

FIGS. 9-11 are schematics illustrating an exemplary method of a surgical procedure using the device of FIG. 1, according to an exemplary embodiment of the invention.

### DESCRIPTION OF THE EMBODIMENTS

Reference will now be made in detail to the exemplary embodiments consistent with the present invention, examples of which are illustrated in the accompanying drawings. Wherever possible, the same reference numbers will be used throughout the drawings to refer to the same or like parts.

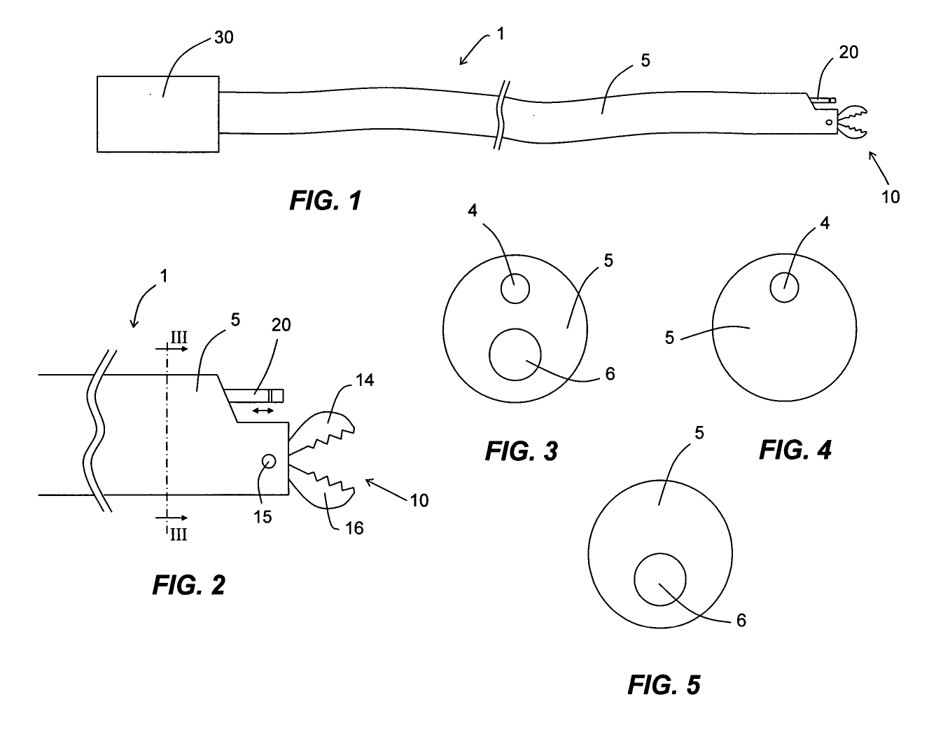

FIG. 1 illustrates an endoscopic medical device 1 (e.g., tissue acquisition device) having a separate hemostatic device for use in, for example, obtaining tissue samples from a patient's body, according to an exemplary embodiment of the invention. While the invention will be described in connection with a particular tissue acquisition device (i.e., biopsy forceps), embodiments of the invention may be used with any other types of tissue acquisition device (e.g., snares, scissors, needles, pincers, knives, knifeneedles, or baskets). Moreover, certain aspects of the inventions may be applied to, or used in connection with, other numerous surgical applications involving tissue cutting that may require a hemostatic treatment.

As shown in FIG. 1, the medical device 1 may comprise a generally tubular body 5 or shaft, a biopsy jaw assembly 10 coupled to the distal portion of the tubular body 5, and a hemostatic device 20 adjacent the jaw assembly 10 in the distal portion of the tubular body 5. The tubular body 5 may be coupled to a suitable actuation member 30 located at the proximal end of the tubular body 5. The actuation member 30 may be connected to the jaw assembly 10 and/or the hemostatic device 20 via a suitable control mechanism, such as, for example, one or more control wires (not shown). In some exemplary embodiments, a separate actuation member may be provided for each of the jaw assembly 10 and the hemostatic device 20.

As best shown in FIG. 2, the jaw assembly 10 may include a pair of jaws 14, 16 having sharp cutting edges or teeth for severing tissue. Actuation of the actuation member may cause the pair of jaws 14, 16 to open and close to sever tissue. In some exemplary embodiments, as shown in FIG. 2, the pair of jaws 14, 16 may be pivotally coupled to the tubular body 5 via a suitable pivot member 15. The jaw assembly 10 and its corresponding actuation member and/or control mechanism may be of any conventional type known in the art.

The hemostatic device 20 may comprise a laser fiber configured to direct a localized laser energy onto the bleeding tissue site, so as to coagulate the bleeding tissue. Alternatively or additionally, the hemostatic device 20 may comprise an applicator, an injection needle, or a spray nozzle configured to apply a therapeutic agent, such as, for example, hemostatic agent, sclerosing agent, and/or coagulating agent, to the surface of the bleeding tissue site. For example, in some exemplary embodiments, epinephrine, ethanol, and hypertonic saline may be used for non-variceal bleeding, and sodium tetradecyl sulfate, sodium morrhuate, ethanolamine oleate, polidocanol, and ethanol may be used for variceal bleeding. Any other known therapeutic agent may be used alternatively or additionally.

The hemostatic device 20 may be axially extendable and/or radially rotatable to facilitate accurate positioning of the device 20 with respect to the tissue to be treated.

Depending on the type of the hemostatic device 20, the jaw assembly 10 may be suitably insulated from the hemostatic device 20 to prevent or minimize the damage to the severed tissue contained in the biopsy forceps.

The tubular body 5 may be sufficiently flexible to traverse through a tortuous body lumen, yet sufficiently stiff to resist compressive force applied thereto during normal operation. The tubular body 5 may or may not be hollow. For example, the tubular body 5 may define at least one lumen 4, 6 along its longitudinal axis to accommodate the jaw assembly 10 and/or the hemostatic device 20, as shown in FIG. 3. In this particular embodiment, at least one of the jaw assembly 10 and the hemostatic device 20 may be separately provided so as permit independent movement in and out of the lumen 4, 6 relative to each other. In an embodiment, the tubular body 5 may be an endoscope defining a plurality of working lumens 4, 6, through which the jaw assembly 10 and/or the hemostatic device 20 may pass.

In some other exemplary embodiments, at least one of the jaw assembly 10 and the hemostatic device 20 may be fixedly connected to the tubular body 5. For example, the jaw assembly 10 may be fixedly connected to the tubular body 5, for example by a pivot pin 15 as shown in FIGS. 1 and 2, and the hemostatic device 20 may be movably arranged within the lumen 4 of the tubular body 5, such as the tubular body 5 shown in FIG. 4. Conversely, the hemostatic device 20 may be fixedly connected to the tubular body 5, and the jaw assembly 10 may be movably arranged within the lumen 4 of the tubular body 5, such as the tubular body 5 shown in FIG. 5. When the movable device (whether it is the jaw assembly 10 or the hemostatic device 20) is not in use, the movable device may be removed from the lumen 4, 6, so that other suitable devices may be inserted into the lumen to reach the tissue site, if desired.

According to another exemplary embodiment, both the jaw assembly 10 and the hemostatic device 20 may be fixedly connected to the tubular body 5. For example, the jaw assembly 10 and the hemostatic device 20 may be integrally formed with the tubular body 5. When the jaw assembly 10 and the hemostatic device 20 are integrally formed, the tubular body 5 may still define one or more lumens or passageways therein.

When the jaw assembly 10 or the hemostatic device 20 is fixedly connected to the tubular body 5, the jaw assembly 10 or the hemostatic device 20 may be configured to extend radially and/or axially, at least to a certain extent, to facilitate positioning relative to the tissue site. This may permit accurate positioning of the jaw assembly 10 and/or the hemostatic device 20 with respect to the target tissue.

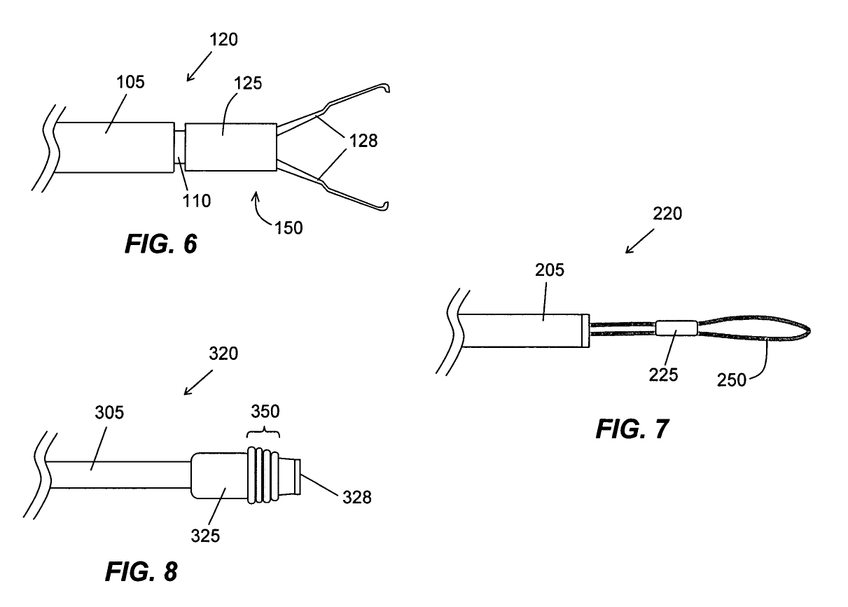

According to some aspects of the invention, the medical device 1 may employ various different types of a hemostatic device. For example, the device 1 may include a clip applicator 120 configured to deploy one or more detachable clips (e.g., hemoclips), as shown in FIG. 6. The applicator 120 may comprise an elongated tube 105 extending from a suitable actuation handle (not shown) and one or more clips 150 disposed proximate the distal end of the tube 105. In some exemplary embodiments, the clip 150 may be detachably coupled to the distal end of the tube 105 via a suitable connecting member 110. The clip 150 may comprise a main body 125 and a pair of jaws 128 pivotally coupled to the main body 125. In an exemplary embodiment, the jaws 128 may have more than two jaws. The jaws 128 may be opened or closed by actuation of the actuation handle. The actuation handle may also cause detachment and/or deployment of the clip 150 from tube 105. The clip 150 may be configured to be re-coupled to the tube 105 so as to permit repositioning or removal of the clip 150.

In another exemplary embodiment, the device 1 may include a loop applicator 220. As shown in FIG. 7, the loop applicator 220 may comprise an elongated tube 205 and a ligation loop 250 detachably disposed proximate the distal end of the tube 205. The proximal end of the elongated tube 205 may be coupled a suitable actuator (not shown) to control the movement of the loop 250. The loop 205 may include a slidable sheath 225 so as to adjust the size and the ligating tension of the loop 250. The loop 250 may be used to ligate a target tissue prior to or after a medical procedure. For example, during a polypectomy procedure, the loop 250 may be placed around a target tissue below a location to be cut, so as to minimize a post-procedure bleeding.

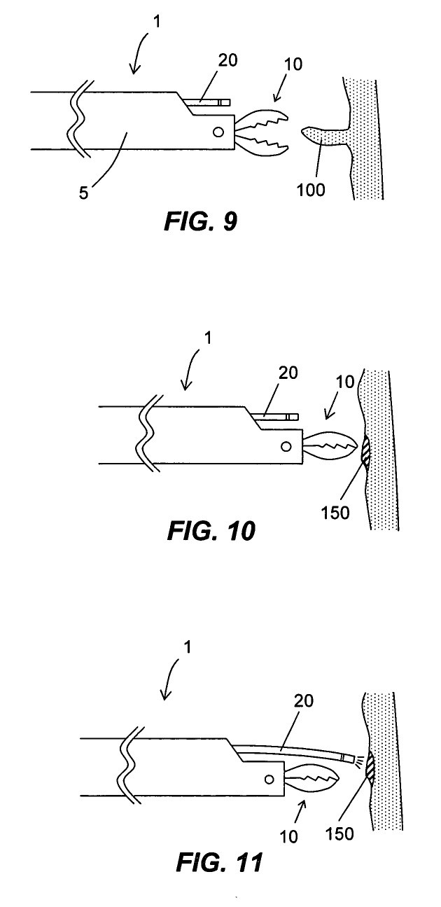

According to still another exemplary embodiment of the invention, the device 1 may include a band applicator 320. As shown in FIG. 9, the applicator 320 may include an elongated member 305 extending from a suitable actuation handle (not shown) and a distal member 325 coupled to the distal end of the elongated member 305. The distal member 325 may include one or more bands 350 or rings that may be placed onto a target tissue to ligate the tissue. The elongated member 305 may define a hollow lumen therethrough, and the distal member 325 may define an opening 328 that may communicate with the hollow lumen of the elongated member 305. The hollow lumen of the elongated member 305 may extend to the actuation handle and connect to a suitable suction source. To place the bands 350 onto a target tissue, according to an exemplary embodiment, the suction source may be turned on and a target tissue may be held by, or suctioned into, the opening 328. Once the target tissue is secured to the opening 328, the band 350 may be placed over the target tissue to ligate the tissue.

Other various types of a hemostatic device may be used alternatively or additionally. For example, in some exemplary embodiments, the hemostatic device 20 may include an electrocautery device configured to allow cauterizing electric current (e.g., bipolar or monopolar current) to flow to the bleeding tissue site to stop the bleeding. In still another exemplary embodiment, the hemostatic device 20 may comprise a gas-induced plasma coagulation device (e.g., argon plasma coagulation device) configured to direct heat energy generated by ionizing the gas onto the bleeding tissue site to coagulate the bleeding tissue. Any other hemostasis device known in the art may also be used additionally or alternatively.

The operation of the medical device 1, according to an exemplary embodiment of the invention, will be described in detail with reference to FIGS. 9-11. While operational aspects of the invention will be described in connection with a particular tissue removal procedure, embodiments of the invention may be applied to other suitable medical procedures, or used in connection with any other suitable medical devices, without departing from the scope of the invention.

As shown in FIG. 9, the device 10 may be inserted into a body lumen of a patient, for example over a suitable guidewire, through a lumen of an endoscope, or via any other conventional method, to position its distal end near the tissue 100 to be removed. Proper positioning of the device 1 may be assisted by any suitable, known visualization method. Once the distal end of the device 10 is properly positioned, the actuation member 30 may be actuated to operate the jaw assembly 10 and to sever the tissue 100 from the target site, as shown in FIG. 10. Severing of the tissue 100 may cause bleeding at the tissue site 150 where the severing occurred. The hemostatic device 20 may be used to stop the bleeding at the tissue site 150, as shown in FIG. 11. It should be understood that any other hemostatic device described above may be used instead. If desired, the hemostatic device 20 may be extended and/or rotated to obtain proper positioning of the device 20 with respect to the bleeding tissue site 150. Once the bleeding stops, the device 1 may be removed from the patient's body or moved to another location to repeat the procedure on another tissue. In certain applications, if desired, one or both of the jaw assembly 10 and the hemostatic device 20 may be removed from the tubular member 5 and replaced with other suitable devices, with the tubular member 5 remaining in place within the patient's body.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device comprising:
an elongated tubular member having a proximal end and a distal end;
a tissue cutting member proximate the distal end of the tubular member; and
a hemostatic member proximate the distal end of the tubular member and adjacent the tissue cutting member,
wherein at least one of the tissue cutting member and the hemostatic member is fixedly connected to the tubular member.

2. The device of claim 1, wherein both of the tissue cutting member and the hemostatic member are fixedly connected to the tubular member.

3. The device of claim 1, wherein one of the tissue cutting member and the hemostatic member is fixedly connected to the tubular member, and the other of the tissue cutting member and the hemostatic member is movably arranged within the tubular member.

4. The device of claim 3, wherein the tubular member defines a lumen extending between the proximal end and the distal end, the lumen being configured to accommodate the other of the tissue cutting member and the hemostatic member.

5. The device of claim 1, wherein the tissue cutting member comprises a tissue acquisition device.

6. The device of claim 5, wherein the tissue acquisition device comprises a biopsy forceps.

7. The device of claim 1, wherein the tissue cutting member comprises at least one of a snare, scissors, pincer, needle, knife, needleknife, and basket.

8. The device of claim 1, wherein the hemostatic member comprises one of a laser fiber probe, a heater probe, an injection needle configured to inject a therapeutic agent, an electrocautery device, a clip applicator, a ligation device configured to apply a band or loop, and a gas-induced plasma probe.

9. The device of claim 1, wherein the elongated member is sufficiently flexible to traverse tortuous anatomy of a patient.

10. A method of performing a medical procedure, comprising:
inserting a medical device into a patient's body, the medical device comprising:
an elongated tubular member having a proximal end and a distal end,
a tissue cutting member proximate the distal end of the tubular member, and
a hemostatic member proximate the distal end of the tubular member and adjacent the tissue cutting member;
wherein at least one of the tissue cutting member and the hemostatic member is fixedly connected to the tubular member;
positioning the distal end of the tubular member near a target site;
severing tissue from the target site with the tissue cutting member; and
stopping bleeding at the target site with the hemostatic member.

11. The method of claim 10, further comprising removing the medical device out of the patient's body.

12. The method of claim 10, wherein both of the tissue cutting member and the hemostatic member are fixedly connected to the tubular member.

13. The method of claim 10, wherein one of the tissue cutting member and the hemostatic member is fixedly connected to the tubular member, and the other of the tissue cutting member and the hemostatic member is movably arranged within the tubular member.

14. The method of claim 13, wherein the tubular member defines a lumen extending between the proximal end and the distal end, the lumen being configured to accommodate the other of the tissue cutting member and the hemostatic member.

15. The method of claim 10, wherein the tissue cutting member comprises a biopsy forceps.

16. The method of claim 10, wherein the tissue cutting member comprises at least one of a snare, scissors, pincer, needle, knife, needleknife, and basket.

17. The method of claim 10, wherein the hemostatic member comprises one of a laser fiber probe, a heater probe, an injection needle configured to inject a therapeutic agent, an electrocautery device, a clip applicator, a ligation device configured to apply a band or loop, and a gas-induced plasma probe.

18. The method of claim 10, wherein stopping bleeding comprises moving the hemostatic member axially relative to the tissue cutting member.
